(19) United States
(12) Patent Application Publication
(10) Pub. No.: US 2025/0009834 A1
(43) Pub. Date: Jan. 9, 2025

(54) COMPOSITION OF BEVERAGE CONCENTRATE CONTAINING CITRIC ACID AND NATURAL ANTIOXIDANT

(71) Applicants: Chanchai Boonla, Nonthaburi (TH); Nattida Chotechuang, Bangkok (TH); Nalinthip Lordumrongkiat, Bangkok (TH); Natcha Madared, Bangkok (TH); Bundit Prachapibal, Bangkok (TH)

(72) Inventors: Chanchai Boonla, Nonthaburi (TH); Nattida Chotechuang, Bangkok (TH); Nalinthip Lordumrongkiat, Bangkok (TH); Natcha Madared, Bangkok (TH); Bundit Prachapibal, Bangkok (TH)

(21) Appl. No.: 18/717,587

(22) PCT Filed: Dec. 6, 2022
(86) PCT No.: PCT/IB2022/061812

§ 371 Date: Jun. 7, 2024

Related U.S. Application Data
(60) Provisional application No. 63/265,054, filed on Dec. 7, 2021.

(51) Int. Cl. A61K 36/88; A23L 2/385; A23L 2/60; A61K 9/00; A61K 31/194; A61K 31/385; A61K 31/7048; A61K 36/48; A61P 13/04; A61P 39/06

(52) U.S. Cl. CPC A61K 36/88; A23L 2/385; A23L 2/60; A61K 9/0095; A61K 31/194; A61K 31/385; A61K 31/7048; A61K 36/48; A61P 13/04; A61P 39/06

(57) ABSTRACT

Aspects in accordance with the present invention pertain to a composition of a beverage concentrate having anti-nephrolithiasis effect, said composition comprising: 1-9.99% by weight of citric acid; 60-90% by weight of a water extract of banana stem; 0.01-1.02% by weight of lipoic acid; 0.001-2.005% by weight of rutin; and 0.01-0.65% by weight of at least one natural antioxidant.

The present disclosure claims priority to the earlier U.S. Provisional Patent Application No. 63/265,054 filed on Dec. 7, 2021, the entire disclosure of which is incorporated into the present disclosure by way of reference.

### FIELD OF THE INVENTION

The present invention relates to beverage compositions, particularly to those having anti-nephrolithiasis effect, and also particularly to those having lifespan-extending and anti-aging effects.

### BACKGROUND OF THE INVENTION

Nephrolith (i.e., kidney stones) in human is essentially consisted of calcium oxalate (CaOx), the genesis of which is attributed to three major risk factors: inadequate fluid intake, hypocitraturia (i.e., low urinary citrate excretion), and high oxidative stress. Inadequate fluid intake, the first factor, may be addressed by adjusting the subject's fluid intake routine. Hypocitraturia, the second factor, may be treated by citrate compounds, particularly potassium citrate. Oxidative stress, the third factor, is reduced by increasing the subject's antioxidant diet.

Notable examples of conventional medicines for treating nephrolithiasis in human are those containing a citrate compound, including Uralyt-U which is based on potassium citrate.

Despite said conventional medicines, nephrolithiasis patients often fail to adhere to the treatment or preventive measures. Said failures are attributed to one or more of the following: the patients' physiological or behavioral resistance against the change of everyday fluid intake routine, the difficulty of observing the recommended antioxidant diet, and the repulsive flavor that is inherent to the citrate compound.

Several prior arts attempted to simplify the treatment and incorporate it into the patient's daily routine by way of nutraceutical compositions, comprising mainly citric acid and/or citrate compounds and extracts of banana stem (musa). A recent example of such prior arts is the U.S. Patent Publication No. US 2021/0128665 A, describing general food compositions of the aforesaid nature with an object to combine the active agents into one composition. This is one attempt to improve the treatment's accessibility; however, none of the prior arts known to the present inventors has effectively solved the problem of patients' non-adherence to the treatment or preventive measures.

As such, there is a strong need to provide a treatment or preventive measure for nephrolithiasis that is capable of addressing the above problem.

### SUMMARY OF THE INVENTION

Embodiments according to the present invention are related to a beverage composition containing citric acid and a natural antioxidant, particularly to those having anti-nephrolithiasis effect.

In the first aspect, an embodiment is a composition of a beverage concentrate. Said composition has anti-nephrolithiasis effect and comprises: 1-9.99% by weight of citric acid; 60-90% by weight of a water extract of banana (Musa sapientum L.) stem; 0.01-1.02% by weight of lipoic acid; 0.001-2.005% by weight of rutin (C₂₇H₃₀O₁₆); and 0.01-0.65% by weight of at least one natural antioxidant.

Preferably, said lipoic acid is alpha-lipoic acid (ALA).

Preferably, the natural antioxidant has colorant property.

Preferably, said composition comprises 0.01-0.20% by weight of the natural antioxidant, said natural antioxidant being derived from sappan (Caesalpinia sappan L.) heartwood.

Preferably, said composition comprises 0.05-0.45% by weight of the natural antioxidant, said natural antioxidant being derived from butterfly pea (Clitoria ternatea L.) flower.

More preferably, said composition comprises 0.01-0.20% by weight of a first natural antioxidant and 0.05-0.45% by weight of a second natural antioxidant, said first antioxidant being derived from sappan (Caesalpinia sappan L.) heartwood and said second antioxidant being derived from butterfly pea (Clitoria ternatea L.) flower.

Preferably, said composition comprises 0.01-25.20% by weight of a non-sugar natural sweetener.

More preferably, said composition comprises 0.01-0.15% by weight of the non-sugar natural sweetener, said non-sugar natural sweetener being stevia.

Also more preferably, said composition comprises 0.01-0.15% by weight of the non-sugar natural sweetener, said non-sugar natural sweetener being sucralose.

Also more preferably, said composition comprises 14.01-25.20% by weight of the non-sugar natural sweetener, said non-sugar natural sweetener being maltitol.

Preferably, said composition comprises 1.01-5.01% by weight of citric acid.

Preferably, said composition comprises 70-85% by weight of the water extract of banana (Musa sapientum L.) stem.

Preferably, said composition comprises 0.01-0.05% by weight of lipoic acid.

Preferably, said composition comprises 0.001-1.005% by weight of rutin (C₂₇H₃₀O₁₆).

In a preferred embodiment, said composition also has anti-aging and lifespan-extending effects.

Even more preferably, said composition comprises 0.05-0.15% by weight of the natural antioxidant derived from sappan (Caesalpinia sappan L.) heartwood.

Even more preferably, said composition comprises 0.15-0.35% by weight of the natural antioxidant derived from butterfly pea (Clitoria ternatea L.) flower.

The above embodiments effectively address the three risk factors of nephrolithiasis, along with the three main factors of non-adherence to the treatment. Such effects are enabled by the composition's flavor and rich contents of citric acid and antioxidants. Surprisingly, the present inventors found that, consumed on a regular basis, the embodiments provide effective contents of citric acid to prevent hypocitraturia and effective contents of antioxidants (mostly polyphenols and flavonoids) to prevent oxidative stress. Moreover, the composition's flavor is conducive to continued daily consumption, thereby addressing the factor of liquid intake as well as the repulsive flavor of potassium citrate drugs which is present in the prior art.

In the second aspect, an embodiment is a beverage prepared from the composition in accordance an embodiment of the first aspect. The preparation is carried out by mixing the composition with water at the ratio of 0.05-0.15 parts of the composition to one part of water (e.g., 0.05-0.15 mL of the composition to 1 mL of water).

Preferably, the composition is mixed with water at a ratio of 0.10-0.15 parts of the composition to one part of water.

Although embodiments of the present invention can exhibit their intended health effects in any form, the present inventors have determined that the concentrate form adapted for mixing with water provides additional utility: Requiring the user to dilute the concentrate to prepare the beverage in its ready-to-drink form increases the user's daily fluid intake, which in turn addresses one of the risk factors. The concentrate form (preferably packaged in a sachet) also reduces the storage space and weight and thus conducive to industrial production and transport. Compared with the powdered form, the concentrate mixes better and easier with water and increases more fluid intake.

In the third aspect, an embodiment is a method for inducing anti-nephrolithiasis effect in a human body by oral ingestion of a beverage prepared from the composition in accordance with any embodiment of the first aspect.

In said induction of anti-nephrolithiasis effect in a human body, it is preferred that at least 1,000 mL of the beverage is orally ingested daily.

In the fourth aspect, an embodiment is a method for treating a human nephrolithiasis patient by oral ingestion of a beverage prepared from the composition in accordance with any embodiment of the first aspect.

In said treatment of the human nephrolithiasis patient, it is preferred that at least 1,000-1,500 mL of the beverage is orally ingested daily.

### BRIEF DESCRIPTION OF DRAWINGS

The principle of the present invention and its advantages will become apparent in the following description, taking into consideration the accompanying drawings in which:

FIG. 1A compares a prior art anti-nephrolithiasis substance with an embodiment in their total antioxidant capacities.

FIG. 1B shows the total phenolic content (TPC) and total flavonoid content (TFC) of an embodiment.

FIG. 2A shows the comparison of seed calcium oxalate monohydrate (COM) crystals aggregation coefficients between distilled water (DW), a bovine serum albumin (BSA), and an embodiment (“Emb”).

FIG. 2B shows the effects of treatment with an embodiment (“Emb”) on the production of intracellular Reactive Oxygen Species (ROS) in HK-2 cells when exposed to H₂O₂ and seed calcium oxalate monohydrate (COM).

FIG. 2C shows the effects of treatment with an embodiment (“Emb”) on the levels of protein carbonyl content in HK-2 cells when exposed to H₂O₂ and seed calcium oxalate monohydrate (COM).

FIG. 2D shows the effects of treatment with an embodiment (“Emb”) on the morphology of HK-2 cells when exposed to H₂O₂ and seed calcium oxalate monohydrate (COM).

FIG. 3A shows the autofluorescent lipofuscin marking the aging in wild type C. elegans nematodes supplied with various embodiments.

FIG. 3B shows the levels of lipofuscin accumulation (AFU) in wild type C. elegans nematodes supplied with various embodiments (“Emb”).

FIG. 3C shows the effects of treatment with an embodiment (“Emb”) on the relative telomere length (RTL) in HK-2 cells when exposed to H₂O₂, sodium oxalate (NaOx) and seed calcium oxalate monohydrate (COM).

FIG. 3D shows the effects of treatment with an embodiment (“Emb”) on the stress-induced premature senescence (SIPS), as indicated by SA-β-gal positivity and p16 upregulation, in HK-2 cells when exposed to H₂O₂, sodium oxalate (NaOx) and seed calcium oxalate monohydrate (COM).

FIG. 4A shows the effects of treatment with an embodiment, in comparison with the effects of treatment with a prior art anti-nephrolithiasis substance, on the appearances of rat kidneys when ethylene glycol (EG) was administered to the rats to induce calcium oxalate nephrolithiasis.

FIG. 4B shows the effects of treatment with an embodiment, in comparison with the effects of treatment with a prior art anti-nephrolithiasis substance, on the weights of rats' kidneys when ethylene glycol (EG) was administered to the rats to induce calcium oxalate nephrolithiasis.

FIG. 5A shows the images obtained from polarized light microscopy and from Yasue straining for comparison between the effects of treatment with an embodiment and the effects of treatment with a prior art anti-nephrolithiasis substance on the growth of calcium oxalate crystals in rat kidneys when ethylene glycol (EG) was administered to the rats to induce calcium oxalate nephrolithiasis.

FIG. 5B shows the comparison between the effects of treatment with an embodiment and the effects of treatment with a prior art anti-nephrolithiasis substance on the number of calcium oxalate (CaOx) crystal deposits in rat kidneys when ethylene glycol (EG) was administered to the rats to induce calcium oxalate nephrolithiasis.

FIG. 6A shows the qualitative and quantitative comparisons between the effects of treatment with an embodiment and the effects of treatment with a prior art anti-nephrolithiasis substance on the intrarenal expressions of 4-hydroxynonenal (4-HNE), as an oxidative stress marker, in rats when ethylene glycol (EG) was administered to the rats to induce calcium oxalate nephrolithiasis.

FIG. 6B shows the comparison between the effects of treatment with an embodiment and the effects of treatment with a prior art anti-nephrolithiasis substance on the urinary oxalate in rats when ethylene glycol (EG) was administered to the rats to induce calcium oxalate nephrolithiasis.

FIG. 6C shows the comparison between the effects of treatment with an embodiment and the effects of treatment with a prior art anti-nephrolithiasis substance on the urinary citrate in rats when ethylene glycol (EG) was administered to the rats to induce calcium oxalate nephrolithiasis.

FIG. 6D shows the comparison between the effects of treatment with an embodiment and the effects of treatment with a prior art anti-nephrolithiasis substance on the urinary uric acid in rats when ethylene glycol (EG) was administered to the rats to induce calcium oxalate nephrolithiasis.

FIG. 6E shows the comparison between the effects of treatment with an embodiment and the effects of treatment with a prior art anti-nephrolithiasis substance on the urinary indole-reacted calcium oxalate crystallization index (iCOCI) in rats when ethylene glycol (EG) was administered to the rats to induce calcium oxalate nephrolithiasis.

FIG. 7A shows the lifespans, represented by survival rates over time, of wild type C. elegans nematodes supplied with various embodiments (“Emb”).

FIG. 7B shows the food intake behavior, represented by pharyngeal pumping rates over time, of wild type C. elegans nematodes supplied with various embodiments (“Emb”).

FIG. 8A shows the levels of urinary total phenolic content (TPC) in the 24-hours urine samples collected from the subjects of Group 1 (“Embodiment”) and Group 2 (“Placebo”) after the Intervention (D10) administered in the Clinical Trials in Healthy Human Subjects.

FIG. 8B shows the levels of urinary total phenolic content (TPC) in the 24-hours urine samples collected from the subjects of Group 1 (“Embodiment”) and Group 2 (“Placebo”) compared between before and after the Intervention (D10) administered in the Clinical Trials in Healthy Human Subjects.

FIG. 9A compares the urinary indole-reacted calcium oxalate crystallization indexes (iCOCI) in the 24-hours urine samples collected from the subjects of Group 2 (“Placebo”) before and after the Intervention administered in the Clinical Trials in Healthy Human Subjects.

FIG. 9B compares the urinary indole-reacted calcium oxalate crystallization indexes (iCOCI) in the 24-hours urine samples collected from the subjects of Group 1 (“Embodiment”) before and after the Intervention administered in the Clinical Trials in Healthy Human Subjects.

FIG. 10 compares the plasma protein carbonyl contents in the blood samples collected from the subjects of Group 1 (“Embodiment”) and Group 2 (“Placebo”) during the PK study phase in the Clinical Trials in Healthy Human Subjects.

FIG. 11 compares the urinary citrate excretions in the urine samples collected from the subjects of Group 1 (“Embodiment”) and Group 2 (“Placebo”) during the PK study phase in the Clinical Trials in Healthy Human Subjects.

FIG. 12 compares the electrical conductivities of the 24-hour urine samples collected from the subjects before and after the 30-day Intervention with an embodiment in the Pilot Trials in Human Nephrolithiasis Patients.

FIG. 13A compares the urinary calcium oxalate crystallization index (COCI) of the 24-hour urine samples collected from the subjects before and after the 30-day Intervention with an embodiment in the Pilot Trials in Human Nephrolithiasis Patients.

FIG. 13B compares the urinary indole-reacted calcium oxalate crystallization index (iCOCI) of the 24-hour urine samples collected from the subjects before and after the 30-day Intervention with an embodiment in the Pilot Trials in Human Nephrolithiasis Patients.

FIG. 14A compares the total antioxidant capacity (TAC) of the 24-hour urine samples collected from the subjects before and after the 30-day Intervention with an embodiment in the Pilot Trials in Human Nephrolithiasis Patients.

FIG. 14B compares the total phenolic content (TPC) in the 24-hour urine samples collected from the subjects before and after the 30-day Intervention with an embodiment in the Pilot Trials in Human Nephrolithiasis Patients.

FIG. 15 compares urinary citrate in the 24-hour urine samples collected from the subjects before and after the 30-day Intervention with an embodiment in the Pilot Trials in Human Nephrolithiasis Patients.

FIG. 16A shows the results of Fourier transform infrared (FTIR) spectroscopy analysis performed upon the nephroliths samples collected from a Trial subject after the Intervention with an embodiment in the Pilot Trials in Human Nephrolithiasis Patients.

FIG. 16B shows the results of Fourier transform infrared (FTIR) spectroscopy analysis performed upon the nephroliths samples collected from another Trial subject after the Intervention with an embodiment in the Pilot Trials in Human Nephrolithiasis Patients.

FIG. 17 shows the satisfaction ratings from the Taste Tasting Survey Trials conducted for an embodiment.

### DETAILED DESCRIPTION OF EMBODIMENTS OF THE INVENTION

It is to be understood that the following detailed description will be directed to embodiments, provided as examples for illustrating the concept of the present invention only. The present invention is in fact not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section.

Unless defined otherwise, all technical and scientific terms used herein have the same meaning as commonly understood by one of ordinary skills in the art to which this invention belongs.

It must be noted that as used herein and in the appended claims, the singular forms “a”, “an”, and “the” include plural referents unless the context clearly dictates otherwise.

The term “about” when used before a numerical designation, e.g., dimensions, time, amount, and such other, including a range, indicates approximations which may vary by (+) or (−) 10%, 5% or 1%, or any sub-range or sub-value there between.

“Comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a device or method consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

“Water extract” refers to the extraction of natural antioxidants from banana stem using water, preferably hot water. In connection with exemplary embodiments, water extraction is carried out by boiling one part of chopped banana stem with one part of water for 1-2 hours. The water extract is then squeezed out from the boiled banana stem, filtered, and stored at −20° C.

The present inventors have carried out the following Trials to ascertain embodiments' health benefits, including anti-aging and lifespan-extending effects, along with the embodiments' safety and capability to behaviorally induce the fluid intake.

In the following Trials, all data are presented as mean±standard deviation (SD) or median (interquartile range, IQR) as appropriate. Two-sample t-test or Mann Whitney test was performed to test the difference between the two groups. One-way ANOVA or Kruskal-Wallis test followed by multiple comparison test was used to test the difference among three or more groups. GraphPad Prism Software version 9 or later was employed for computation and graphs. P<0.05 was considered statistically significant.

In the following Trials, embodiments pertaining to beverage concentrates embodied the exemplary composition shown in Table 1 below.

### Table 1 Shows an Exemplary Composition of a Preferred Embodiment:

| | |
|---|---|
| Citric acid | 1.700% by weight |
| Water extract of banana (Musa sapientum L.) stem | 82.851% by weight |
| Alpha-lipoic acid (ALA) | 0.041% by weight |
| Rutin (C₂₇H₃₀O₁₆) | 0.002% by weight |
| Sappan (Caesalpinia sappan L.) heartwood powder | 0.080% by weight |
| Butterfly pea (Clitoria ternatea L.) flower powder | 0.250% by weight |
| Natural stevia | 0.083% by weight |
| Maltitol | 14.910% by weight |
| Sucralose | 0.083% by weight |

### Nutrition Contents

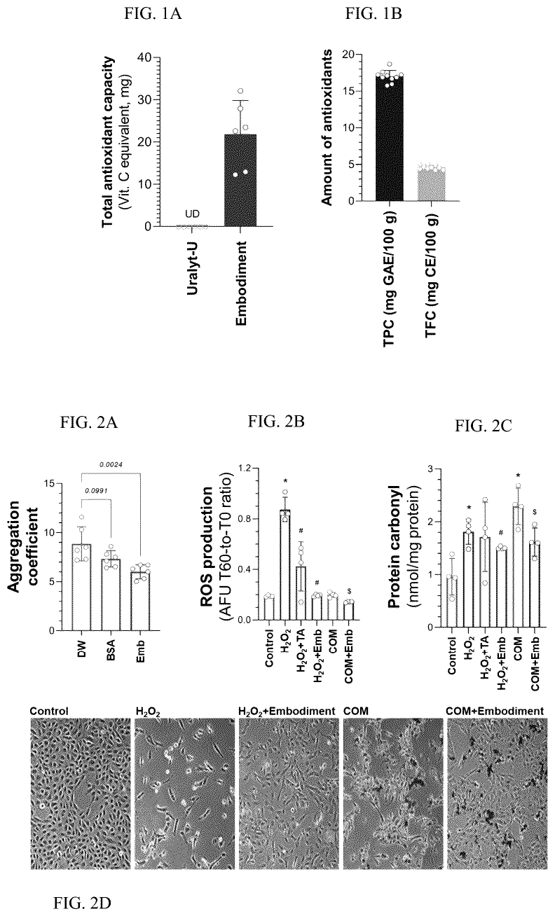

Tests for nutrition contents were carried out upon a 55-mL sample of a beverage concentrate of an exemplary composition (see Table 1 above). Total antioxidant capacity was 21.87±7.96 mg vitamin C equivalent antioxidant capacity per the same volume (Total antioxidant capacity in the Uralyt-U potassium citrate drug was undetectable.) (FIG. 1A). Total phenolic content (TPC) was 19.99±0.83 mg gallic acid equivalent/100 g (FIG. 1B). Total flavonoid content was 4.59±0.31 mg catechin equivalent/100 g (FIG. 1B). Total energy was of 25 kcal. Protein, fat, and cholesterol were not detected. Total carbohydrate was 6 g. Sugar was less than 1 g, and sodium was 10 mg. Potassium content was of 240 mg/100 g. Soluble and insoluble dietary fibers were 0.74 and 0.43 g/100 g, respectively. The pH of was 3.4. Rutin (C₂₇H₃₀O₁₆) content (0.002% by weight) was measured by the thin layer chromatography.

### In-Vitro Trials

In the following in-vitro trials, all “%” marks represented percentages by weight. An embodiment of “100%” concentration was prepared by mixing 60 mL of a beverage concentrate of an exemplary composition (see Table 1 above) with 440 mL of distilled water, equivalent to a ratio of 0.136 parts of the exemplary composition to one part of water. An embodiment of a concentration lower than 100% was prepared by further diluting said embodiment of 100% concentration with distilled water by a conventionally known dilution means that is appropriate for food and beverages. All the mixtures concerned were conventionally mixed to achieve effective uniformity for food and beverages.

### CaOx Aggregation Inhibition and Antioxidant Function

Seed calcium oxalate monohydrate (COM) crystals were prepared and diluted to 50 mg/mL in 0.05 M Tris/0.15 NaCl, pH 6.5. An embodiment having the 100% concentration, or bovine serum albumin (BSA) or distilled water (blank) (200 μL) was added to the freshly prepared working seed COM suspension (2 mL). After mixing, absorbance at 620 nm was measured as baseline (AT₀). After incubating at 37° C. for 10 min. absorbance was measured again (AT₁₀). The aggregation coefficient (AC) was calculated from: AC=((AT₀−AT₁₀)/10)×1,000. Higher AC value indicates higher COM aggregation.

Said embodiment (marked as “Emb” in FIG. 2A) significantly inhibited aggregation of seed COM crystals, but BSA (1 mg/mL) did not. Citric acid (a positive control) strongly inhibited COM aggregation.

### Cell Line and In-Vitro Cytotoxicity

Human kidney (HK)-2 cells (American Type Culture Collection) were maintained in Dulbecco's Modified Eagle Medium (DMEM) supplemented with 10% fetal bovine serum, 1% Pen-Strep, under 37° C., 5% CO₂, and 95% humidity. Cytotoxicity of embodiments in HK-2 cells was assessed by 3-(4,5-dimethylthiazol-2yl)-2,5-diphenyltetrazolium bromide (MTT) assay. HK-2 cells were seeded (200 cells/well), grown overnight in 96-well plate, and treated with varied concentrations of embodiments (0% as control, 1.26%, 2.52%, 5.04%, 10.06%, 20.12%, 40.24%, 50.30%, 80.32% and 100%) for 24 h. After washing, MTT solution was added, incubated for 1 h, and then discarded. Dimethyl sulfoxide was added to solubilize formazan crystals. Absorption at 570 nm was measured. Cell viability (%) was calculated using untreated cells as control (100% viability).

An embodiment having the 10.06% concentration exhibited the highest anti-oxidative activity with the lowest cytotoxicity. An embodiment of the same concentration was selected for the subsequent intracellular reactive oxygen species (ROS) determination and protein carbonylation measurement.

### Intracellular Reactive Oxygen Species (ROS) Determination

Inhibition of intracellular ROS production by embodiments having the 10.06% concentration was determined by dichlorodihydro-fluorescein diacetate (DCFH-DA) assay in 96-well plate. HK-2 cells were combined with 0.1 M DCFH-DA solution and incubated at 37° C. for 30 min. After washing, cells were challenged with H₂O₂ or calcium oxalate monohydrate (COM) (with and without said embodiments). Fluorescent intensity (excited at 485 nm and emitted at 535 nm) was measured at the beginning (T₀) and at 60 min (T₆₀). Arbitrary fluorescent unit (AFU), indicated the level of ROS generation, was calculated from: AFU=T₆₀/T₀.

ROS production in HK-2 exposed to H₂O₂ and calcium oxalate monohydrate (COM) was significantly higher in comparison with the control but was significantly lower than the control after co-treatment with said embodiments (marked as “Emb” in FIG. 2B).

### Protein Carbonylation Measurement

The treated HK-2 cells (in 100 mm dish) were lysed by radioimmunoprecipitation assay buffer containing protease inhibitor cocktail. Total protein concentration was measured by Bradford assay. Cell lysate was incubated with 10 mM dinitrophenylhydrazine (DNPH) solution or 2 N HCl for 1 h in the dark. Cold 20% trichloroacetic acid was added and incubated for 10 min on ice. Pellet was collected by centrifugation, washed with ethyl acetate:ethanol (1:1), and re-dissolved with 6 M guanidine HCl. Absorbance (A) at 375 nm was measured. Protein carbonyl content normalized by total protein concentration was calculated from: ((A_{DNPH}−A_{HCl})×45.45)/protein concentration.

Embodiments having the 10.06% concentration (marked as “Emb” in FIG. 2C) significantly reduced levels of protein carbonyl content in HK-2 exposed to H₂O₂ and COM.

Exposure of HK-2 to H₂O₂ (1,000 μM) and COM (150 μg/cm²) caused cell morphology change and increased cell death. In conclusion, co-treatment with said embodiment prevented morphological change, recovered cells from apoptosis, and restored cell proliferation (FIG. 2D).

### Inhibition of Telomere Attrition, p16 Upregulation, and Premature Senescence in HK-2 Cells

The relative telomere length (RTL) was measured in HK-2 cells treated with H₂O₂ (25 μM), sodium oxalate (NaOx, 900 μM) and COM (25 μg/cm²) with or without embodiments of 10.06% concentration. The RTL was determined by real-time quantitative polymerase chain reaction. It is calculated based on the ratio of copy number of telomeric repeat to copy number of single-copy gene (36B4 gene) that is proportionated to the average telomere length. The used primers were: Telomere (forward) 5′-CGGTTTGTTTGGGTTTGGGTTTGGGTTTGGGTTTGGGTT-3′, Telomere (reverse) 5′-GGCTTGCCTTACCCTTACCCTTACCCTTACCCTTACCCT-3′, 34B4 (forward) 5′-CAGCAAGTGGGAAGGTGTAATC C-3′, and 36B4 (reverse) 5′-CCCATTCTATCATCAACGGGTACAA-3′. PCR was amplified at 95° C. for 10 min, followed by 40 cycles of 95° C. for 15 sec and 54° C. for 1 min.

HK-2 cells grown on coverslip were treated with H₂O₂, NaOx, and COM with and without embodiments of 10.06% concentration for 72 h for an induction of stress-induced premature senescence (SIPS) or premature cellular aging. After treatment, cells were fixed and stained with the freshly prepared X-gal solution (Vivantis, Malaysia) for 12-16 h, washed, permeabilized with 0.1% Triton X-100 (Amresco) for 3 min, and blocked for nonspecific binding with 1% normal horse serum (Gibco) at 37° C. for 1 h. Cells were then incubated with 1:10,000 p16 primary antibody (ab108349, Abcam) at 4° C. overnight, followed by 1:10,000 Alexa Fluor® 488-conjugated secondary antibody (Cell Signaling Technology) at 37° C. for 30 min. The stained coverslip was mounted with Fluoroshield mounting medium with DAPI (Abcam). SA-β-gal positive senescent cells (blue) and p16-expressing cells (green) were visualized and imaged using the EVOS FL Auto 2 imaging system (Thermo Scientific).

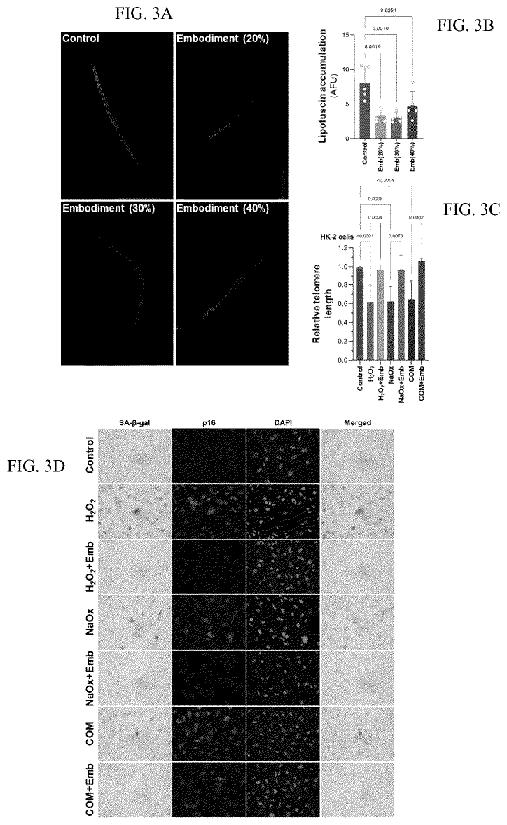

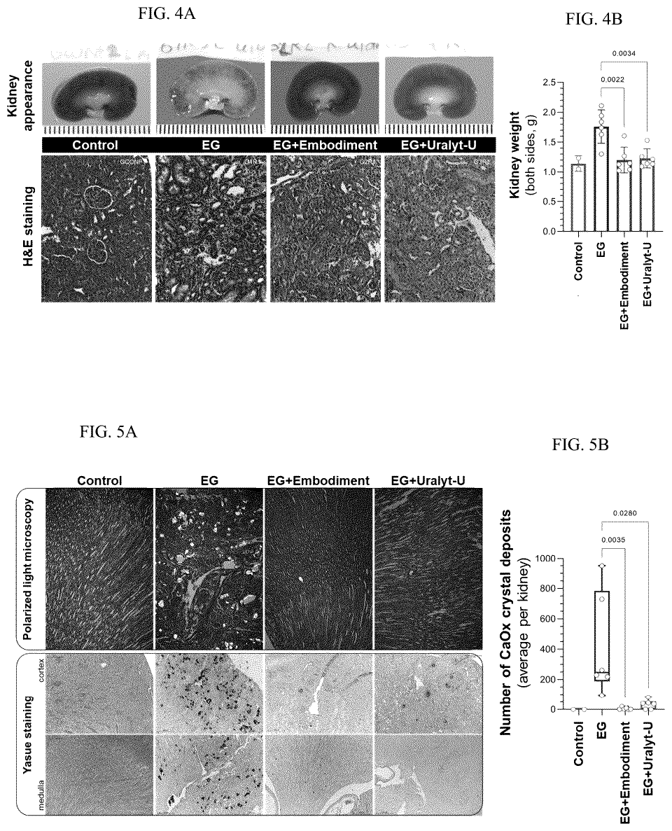

RTL of HK-2 treated with H₂O₂, NaOx, and COM was significantly shorter than that of untreated control (FIG. 3C). Co-treatment with embodiments of 10.06% concentration (marked in FIG. 4C as “Emb”) significantly impeded the shortening of telomere in HK-2 treated with H₂O₂, NaOx, and COM.

Double staining of SA-β-gal and p16 demonstrated that H₂O₂, NaOx, and COM induced SIPS, as indicated by increased proportion of SA-β-gal positive cells, and p16 upregulation in HK-2 cells (FIG. 3D). p16 was intensively labeled in those SA-β-gal positive cells. Co-treatment with embodiments of 10.06% concentration (marked in FIG. 3D as “Emb”) significantly inhibited SIPS (decreased SA-β-gal positivity) and p16 upregulation in HK-2 exposed to H₂O₂, NaOx, and COM (FIG. 3D). In summary, this in-vitro cell model indicated that the embodiments of 10.06% concentration (marked in FIG. 3D as “Emb”) effectively inhibited the stress-induced premature aging in human kidney cells, at least in part, through the inhibition of telomere shortening.

### In-Vivo Trials

In the following in-vivo trials, all “%” marks represented percentages by weight. An embodiment of “100%” concentration was prepared by mixing 60 mL of a beverage concentrate of an exemplary composition (see Table 1 above) with 440 mL of distilled water, equivalent to a ratio of 0.136 parts of the exemplary composition to one part of water. An embodiment of a concentration lower than 100% was prepared by further diluting said embodiment of 100% concentration with distilled water by a conventionally known dilution means that is appropriate for food and beverages. All the mixtures concerned were conventionally mixed to achieve effective uniformity for food and beverages. Embodiments of which the concentrations are not specified and not inferable from the prior mentioning, is presumed to have the 100% concentration.

### In-Vivo Cytotoxicity

In vivo acute toxicity in mice of an embodiment of a beverage concentrate having an exemplary composition as shown on Table 1 was tested by the Medicinal Plant Research Institute, Ministry of Public Health, Thailand, by orally administering water (control group) and said embodiments to mice at the dose of 20 mL/kg (n=10 per group). Mice were observed for 14 days before necropsy. Gross lesions in visceral organs were examined and compared between the embodiments and control groups.

In vivo acute toxicity testing revealed that all mice administered by said embodiments survived until the end of the experiment (14 days). No gross lesions were found in the visceral organs both in the embodiment-administered and control mice. Therefore, LD₅₀ (50% lethal dose) of the embodiment was >20 mL/kg.

### Inhibition of CaOx Crystal Deposits in Rats' Kidneys

Ethylene glycol (EG) (1% v/v) supplemented in drinking water (free access ad libitum, for 35 days) was used for induction of CaOx nephrolithiasis in rats. Effects of EG, embodiments having the 100% concentration, Uralyt-U (a conventional medicine based on potassium citrate), and their combinations were observed. Particularly, Male Wistar rats (6-8 weeks old, 180-300 g) were divided into four groups including EG (n=6), EG+Embodiment (n=6), EG+Uralyt-U (n=6), and normal control (n=2). All rats were housed in stainless-steel cages at 25° C., 12:12 h light-dark cycle. The two control rats were purposely used only for comparison of CaOx deposit in the rats' kidneys and immunohistochemistry (see further below), not for urine chemistry. Embodiments and Uralyt-U were force-fed twice daily (morning and evening) at total citrate dose of 2 mEq per day. 24-h urine specimens were collected at the end of intervention (Day 35) using thymol as preservative. Rats were anesthetized with isoflurane and sacrificed. Both kidneys were removed, cut, and fixed in 10% neutral formalin buffer for histology study.

All rats were purchased from the National Laboratory Animal Center, Salaya Campus Mahidol University, Nakhonpathom, Thailand. The experimental procedures were conducted in accordance with the guidelines for experimental animals by National Research Council of Thailand and approved by the Institutional Animal Care & Use Committee (IACUC), Faculty of Medicine, Chulalongkorn University (No. 022/2560). The study was carried out in compliance with the ARRIVE guidelines.

The biochemical profile of post-intervention 24-h urine samples (Day 35) obtained from experimental rats is shown in below Table 2. Kidneys of EG rats were enlarged and pale (FIG. 4A), and their weights were significantly greater than that of EG+100% concentration Embodiment and EG+Uralyt-U rats (FIG. 4B).

### Table 2 Shows Characteristics of 24-h Urine (Day 35) of the Experimental Rats.

| | Control | EG | EG + Embodiment | EG + Uralyt-U |
|---|---|---|---|---|
| Number of rats | 2 | 6 | 6 | 6 |
| Body weight at Day 0 (g) | 223.0 ± 7.7 | 195.4 ± 21.3 | 214.1 ± 36.3 | 203.9 ± 25.4 |
| Body weight at Day 35 (g) | 371.5 ± 15.8 | 328.2 ± 30.4 | 352.8 ± 40.0 | 349.1 ± 36.1 |
| Urine volume (mL) | 23.7 ± 3.2 | 42.2 ± 18.6 | 34.1 ± 12.8 | 39.6 ± 20.9 |
| Urine pH | 8.0 ± 0.5 | 7.7 ± 0.8 | 7.4 ± 0.6 | 8.0 ± 0.6 |
| Urine specific gravity | 1.030 ± 0 | 1.170 ± 0.3 | 1.024 ± 0 | 1.027 ± 0 |
| Urine creatinine (mg/day) | 9.3 ± 5.0 | 5.1 ± 2.0 | 7.0 ± 4.1 | 5.9 ± 5.0 |
| Urine total protein (mg/day) | 10.7 ± 1.2 | 12.4 ± 3.9 | 13.6 ± 2.6 | 16.3 ± 3.4 |

### CaOx Deposition in Renal Sections by Polarized Microscope

Formalin-fixed paraffin-embedded rat kidney sections were prepared by automated tissue processor. Hematoxylin and eosin (H&E) staining was performed according to conventional procedures. Birefringent CaOx crystal deposits in H&E-stained sections were visualized using polarized light microscope (OLYMPUS BX50).

Birefringent CaOx crystal deposits were markedly observed in renal sections of EG rats, but they were not found in kidney sections of normal control rats (FIG. 5A). These CaOx deposits were almost disappeared in renal sections of EG+100% concentration Embodiment and EG+Uralyt-U rats. These data indicated that the 100% concentration embodiment and Uralyt-U effectively inhibited CaOx stone formation in the rat model.

### Yasue Staining for CaOx Histochemistry

Renal tissue sections were deparaffinized, rehydrated, submerged in 5% acetic acid for 30 min to remove calcium phosphate and calcium carbonate, and washed with distilled water. Sections were incubated with 5% AgNO₃ for 12 min, washed, and incubated with saturated rubeanic acid in 10% ammonium for 1 min. The stained sections were rinsed with 50% ethanol, washed with distilled water, dehydrated, cleared, and mounted. Black CaOx precipitates were visualized under light microscope

Yasue staining confirmed that CaOx crystals (black precipitates) were largely accumulated in the kidneys of EG rats (both in cortex and medulla), but this CaOx deposition was remarkably inhibited by the 100% concentration Embodiment and Uralyt-U treatments (FIG. 5A). Number of black CaOx precipitates in kidney sections of EG+100% concentration Embodiment and EG+Uralyt-U rats were significantly lower than in EG rats (FIG. 5B).

### Immunohistochemical Staining

After deparaffinization and rehydration, antigen retrieval was performed by boiling in sodium citrate buffer using microwave. Endogenous H₂O₂ was inactivated by incubating with 0.3% H₂O₂ for 30 min. Nonspecific binding was blocked by incubating with normal horse serum for 20 min. Sections were then incubated with 1:1,000 4-hydroxynonenal (4-HNE) (ab46545, Abcam) primary antibody at 4° C. overnight followed by incubation with secondary antibody at 37° C. for 30 min. After washing, sections were incubated with ABC reagent (VECTASTAIN® ABC kit) for 30 min, soaked in 1% diaminobenzidine staining reagent for 5 min, and counterstained with hematoxylin for 5 min. Finally, sections were dehydrated, cleared, mounted, and visualized under light microscope.

H&E staining revealed that kidneys of EG rats had a robust sign of inflammation, whereas kidneys of EG+100% concentration Embodiment and EG+Uralyt-U rats appeared to be normal similar to that observed in normal control rats (FIG. 4A).

### Reduction of Oxidative Stress by Embodiment in Rat Renal Tissues Indicated by Reduced Expression of 4-Hydroxynonenal (4-HNE)

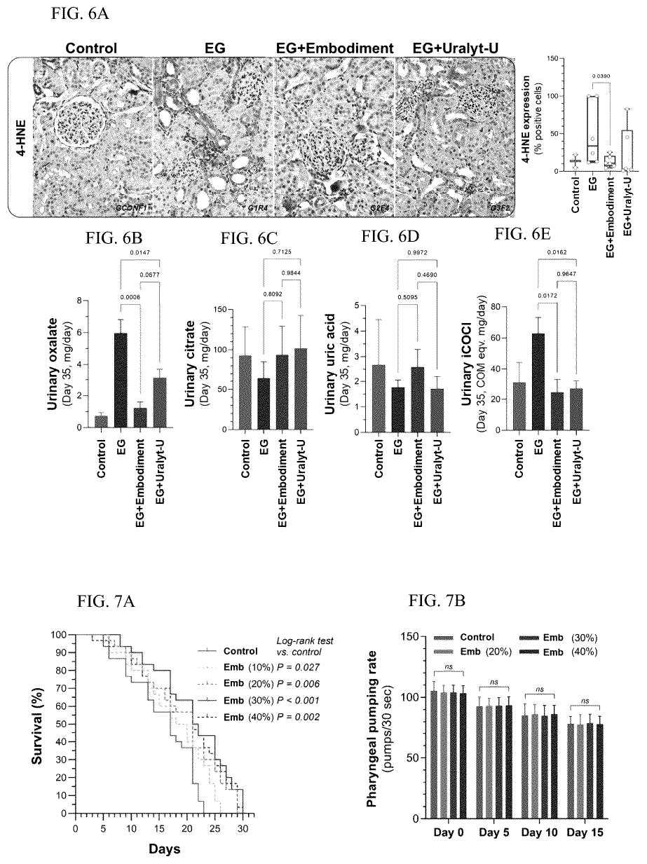

Intrarenal expression of 4-HNE, as an oxidative stress marker, was markedly increased in EG rats compared with normal control rats (FIG. 6A). Overall, 4-HNE expression in kidneys of EG+100% Concentration Embodiment and EG+Uralyt-U rats were lower than EG rats, but quantitatively, proportion of 4-HNE positive cells only in EG+Embodiment rats was significantly lower than that in EG rats (FIG. 6A).

Reduction of Urinary Citrate, Oxalate and Indole-Reacted Calcium Oxalate Crystallization Index (iCOCI) Levels

Urinary level of citrate was determined by high performance liquid chromatography (HPLC) (Varian, USA) using ROA-organic Acid H⁺ column (300×7.8 mm) (Phenomenex, USA), eluted by 5 mM H₂SO₄ at flow rate of 0.5 mL/min. Urinary oxalate and uric acid were measured by capillary electrophoresis (Beckman Counter) separated at 25° C. with voltage of −20 kV. Urinary iCOCI test was performed according to More-Krong et al., Sci Rep. 10 (1), 8334 (2020) which is incorporated by reference.

Urinary oxalate was elevated in EG rats relative to control rats. This urinary oxalate elevation was significantly reduced after treatments with 100% concentration Embodiment and Uralyt-U (at total citrate dose of 2 mEq per day for both) (FIG. 6B). By contrast, urinary citrate was decreased in EG rats relative to control rats. Although treatments with said Embodiment and Uralyt-U did not significantly increase urinary citrate levels relative to EG rats, but they could restore urinary citrate levels to the normal level that found in normal control rats (FIG. 6C). Levels of urinary uric acid compared among EG, EG+Embodiment, and EG+Uralyt-U groups were not significantly different (FIG. 6D). Similar to urinary oxalate, urinary iCOCI levels of EG+Embodiment and EG+Uralyt-U rats were significantly lower than that of EG rats (FIG. 6E).

C. elegans Lifespan Extension

The C. elegans wild-type strain Bristol N2 and its laboratory food source E. coli OP50 were obtained from the Caenorhabditis Genetics Center, University of Minnesota, USA. They were grown and maintained in nematode growth medium (NGM) agar plates with a lawn of E. coli OP50 at 20° C. All experiments were conducted in age synchronized young adult worms.

Analysis of lifespan was performed in liquid media. Briefly, 10 age-synchronized young adult nematodes were transferred into a 24-well plate with M9 buffer along with E. coli OP50 and 5-Fluoro-2′-deoxyuridine for preventing progeny production. Embodiments of 20.12%, 30.18%, and 40.24% concentrations were added using distilled water as vehicle control. The alive nematodes were counted every 24 h. Nematodes were considered dead when they did not respond to gentle prodding using the platinum loop.

Pharyngeal pumping (indicator of food intake ability) was measured in young adult staged nematodes (˜10) compared between control and embodiment supplementation, by monitoring the pharyngeal contraction for 30 sec in every 24 h under the stereomicroscope (Motic SMZ-171) on Day 0 (before supplementation), Day 5, Day 10, and Day 15.

Accumulation of lipofuscin in the wild type nematodes after supplementation with embodiments of 20.12%, 30.18%, and 40.24% concentrations for 5 days were monitored. The E. coli OP50 fed worms were used as control. After intervention, worms were washed using M9 buffer several times, and placed on a glass slide into a drop of sodium azide. Fluorescent imaging was performed using ZEISS LSM 700 confocal microscope. The images were further analyzed using Image J software, and the fluorescence intensity was presented as arbitrary units (AU).

Supplementation with embodiments of 10.06%-40.24% concentrations (marked as “Emb” in FIG. 7A) significantly extended maximum lifespan of wild type C. elegans. The highest lifespan-extending effect was found at the concentration of 30.18% that increased the median survival of nematode from 16.0 days (in control) to 21.5 days (34.4% increase). The median survival of nematodes supplemented with embodiments of 10.06%, 20.12%, and 40.24% concentrations (marked as “Emb” in FIG. 7A) were significantly prolonged to 18.5 (15.6% increase), 19.5 (21.9% increase) and 21.0 (32.3% increase) days, respectively. The findings indicated that the embodiments effectively promoted the longevity in nematode model.

Food intake behavior, as indicated by pharyngeal pumping rates, at Day 0, 5, 10, and 15 were not significantly different between nematodes supplemented with the embodiments of 20.12%, 30.18% and 40.24% concentrations (marked as “Emb” in FIG. 7B) and non-supplemented control nematodes (FIG. 7B). The level of autofluorescent lipofuscin (aging marker) was monitored inside the nematodes after supplementations with embodiments of 20.12%, 30.18% and 40.24% concentrations (marked as “Embodiments” in FIG. 3A). Embodiment supplementations at all tested concentrations (20.12%, 30.18% and 40.24%) significantly reduced the levels of lipofuscin accumulation in nematodes compared with control (FIG. 3B). These data indicated that the embodiments efficiently delayed aging onset in nematode model.

### Clinical Trials in Healthy Human Subjects

### Description of the Trial

Clinical Trial in which the subjects were healthy human volunteers was conducted. The screening was in accordance with the following criteria.

The inclusion criteria were: (1) subjects of all genders, aged 18 to 55 years; (2) subjects having body mass index (BMI) of 18-25 kg/m²; (3) subjects who were healthy by medical history, physical examination and vital signs; (4) subjects whose laboratory values of blood tests including complete blood count, fasting blood sugar, blood urea nitrogen, serum creatinine, alkaline phosphatase, ALT, AST, total bilirubin, direct bilirubin, albumin, and electrolytes were within the normal range or showing no clinically significant abnormalities as confirmed by the clinical investigator; and (5) subjects who were informed of the Trial's requirements and voluntarily signed and dated Informed Consent as approved by the Independent Ethic Committee (IEC)/Institutional Review Board (IRB), prior to the initiation of any screening or study-specific procedures.

Further inclusion criteria were applied to female subjects specifically. Female subjects who were in childbearing potential must have serum j-HCG negative and agreed to use an acceptable birth control method throughout the Trial; the acceptable birth control method was defined as a barrier method of contraception (including condoms, intrauterine device (IUD) and diaphragm with spermicidal agent) or a total abstinence from sexual intercourse; hormonal contraceptives were not acceptable. Female subjects who were in childbearing potential must agree not to become pregnant for the entire Trial and must have a negative result for urine pregnancy test performing prior to every dosing. Eligible female subjects included those with non-childbearing potential, defined as female subjects with hysterectomy, both ovaries removed, surgically sterilized or postmenopausal (for at least 12 consecutive months of amenorrhea).

The exclusion criteria were: (1) subjects with history/evidence of allergy or hypersensitivity to banana or similar products; (2) subjects with a history of any illness that, in the opinion of the clinical investigator, might confound the result of the study or pose an additional risk in administrating embodiments to the subjects, including but not limited to a history of having urinary stone disease or having symptoms suspected of urinary stone disease, a history of relevant drug or food allergies, a history of cardiovascular, gastrointestinal, central nervous system disease, renal and hepatic impairment, malignancy, a history or presence of clinically significant illness, and a history of mental illness that may affect compliance with study requirements; (3) subjects with a history of heavy smoking (more than 10 cigarettes per day) or moderate smoking (less than 10 cigarettes per day) and cannot abstain from smoking at least one day before the Trial and until the completion thereof; (4) subjects with a history of being alcoholics (more than 2 years) or moderate drinkers (more than 3 drinks per day—one is equal to one unit of alcohol: one glass of wine, half pine of beer or one measure of spirit), or subjects with a history of any drug abuse; (5) subject who had received any medical prescription within 14 days before the Trial; (6) female subjects who were pregnant or breastfeeding; and (7) subjects who had been participating or in any investigational drug study, or had donated their blood within 2 months before the screening.

Pursuant to said inclusion and exclusion criteria, from 56 candidates 48 eligible healthy human subjects were selected and then further randomized into two groups of 24 subjects (n). The first group (Group 1, n=24) was instructed to orally ingest the embodiments according to a certain regimen. The second group (Group 2, n=24) was instructed to orally ingest placebos according to the same regimen followed by Group 1.

The embodiment contained in each dosage was prepared by mixing 60 mL of a beverage concentrate of an exemplary composition (see Table 1 above) with 440 mL of reverse-osmosis (RO) water, equivalent to a ratio of 0.136 parts of the exemplary composition to one part of water.

The Trials consisted of four study phases: the pre-pharmacokinetic (PK) study phase, the PK study phase, the normal-lifestyle phase, and the post-PK study phase. The pre-PK and PK phases took place in the first four Days wherein all subjects were admitted to a clinical trial research center in which their lifestyles, especially diets, were controlled; in the normal-lifestyle phase and the post-PK study phase all subjects were discharged from the research center and instructed to conduct their lives normally, except to orally ingest the prescribed embodiments or placebos, as the case maybe, according to the same regimen. Towards the end of normal-lifestyle phase, the overlapping post-PK study phase began. During the post-PK study phase the subjects were instructed to collect their urine samples according to the respective instructions to be described below.

In Days 1 and 2 (i.e., the pre-PK study phase), the subjects were yet to ingest an embodiment or a placebo; instead, they were instructed to collect their urine samples throughout those Days.

Subsequently in Days 3 and 4 (i.e., the PK study phase), the “Intervention” was administered: the subjects were instructed to orally ingest two dosages of embodiments (Group 1) or placebos (Group 2), each dosage containing 500 mL of embodiment/placebo, immediately after the breakfast and dinner of Day 3. From both Groups blood samples were collected according to the following schedule: before the breakfast and dinner of Day 3 (i.e., before ingesting each dosage of embodiment/placebo); 15, 30, 45, 60, and 120 minutes after the breakfast and dinner of Day 3 (i.e., after ingesting each dosage of embodiment/placebo); and the early morning (about 07:00) of Day 4. Total times of blood sample collected in the PK study phase were therefore 13 times per subject regardless of their Group. Further, from both Groups urine samples were collected according to the following schedule: before the breakfast and dinner of Day 3 (i.e., before ingesting each dosage of embodiment/placebo); every 2 hours after the breakfast and dinner of Day 3 (i.e., after ingesting each dosage of embodiment/placebo) including the bedtime during which the subjects were woken to collect urine samples every 2 hours; the first morning urine of Day 4; and finally about 08:00 (morning) of Day 4. Afterwards the subjects were discharged from the clinical trial research center and entered the normal-lifestyle phase.

In Days 4-9 during which the subjects underwent the normal-lifestyle phase, they were instructed to conduct their lives normally, with the following Intervention being maintained: starting from Day 4, the subjects were instructed to orally ingest two dosages of embodiments (Group 1) or placebos (Group 2), each dosage containing 500 mL of embodiment/placebo, immediately after every breakfast and dinner until the dinner of Day 9.

Finally, the post-PK study phase began in Day 9. The subjects of Groups 1 and 2 alike were instructed to collect their urine samples over a 24-hour interval within the span of Days 9 and 10. For example, a subject who chose to collect their first urine sample on 06:00 (morning) of Day 9 would continue said collection until 06:00 (morning) of Day 10. Then the subjects were instructed to deliver the urine samples collected thus to the clinical trial research center by Day 10. Said sample delivery concluded the Trial in healthy human subjects.

All the blood and urine samples collected from the Trial (1,248 samples in total) were preserved and subsequently analyzed.

### Analysis Results

The analysis results of 24-hour urine's volumes, pH, specific gravity, and creatinine are shown in Table 3 below.

### Table 3 Shows the Results of Analyses Upon the Subjects' 24-Hour Urine Pursuant to the Trial.

| | Parameter | | | |
|---|---|---|---|---|
| | Group 1 (ingesting embodiments) | | Group 2 (ingesting placebos) | |
| | Before the | After the | Before the | After the |
| 24-hour urine | Intervention | Intervention | Intervention | Intervention |
| samples | (Day 1-2) | (Day 9-10) | (Day 1-2) | (Day 9-10) |
| 24-hour urine | 1,333 ± 640 | 1,877 ± 779⁽¹⁾ | 1,466 ± 783 | 1,869 ± 705⁽²⁾ |
| volume (mL) | | | | |
| 24-hour urine pH | 6.5 ± 0.4 | 6.6 ± 0.5 | 6.5 ± 0.5 | 6.5 ± 0.5 |
| 24-hour urine | 1.013 ± 0.007 | 1.009 ± 0.006 | 1.011 ± 0.006 | 1.006 ± 0.004 |
| specific gravity | | | | |
| 24-hour urine | 0.77 ± 0.54 | 1.10 ± 0.76 | 0.61 ± 0.37 | 0.81 ± 0.55 |
| creatinine (g/day) | | | | |

Remarks on Table 3:
⁽¹⁾P < 0.001 vs. the embodiment (before the Intervention);
⁽²⁾P = 0.009 vs. the placebo (before the Intervention).

After the Trial's Intervention, both Group 1 (the Intervention involving ingesting embodiments) and Group 2 (the Intervention involving ingesting placebos) exhibited higher 24-hour urine volume and lower urine specific gravity. Particularly, after the Intervention, Groups 1 and 2 showed the increases of the 24-hour urine volume from 1,333 to 1,877 mL and from 1,466 to 1,869 mL respectively; and the decreases of the 24-hour urine specific gravity from 1.013 to 1.009 and from 1.011 to 1.006 respectively. With reference to Casa et al., J Athl Train 35 (2) (2000) 212-224, a human's urine should have less than 1.010 specific gravity to be considered well hydrated. Accordingly, the Interventions administered to both Groups improved the subjects' hydration level from “minimally hydrated” to “well hydrated”.

After the Intervention with embodiments, the subjects of Group 1 exhibited a higher 24-hour urine pH, particularly from 6.5 to 6.6. The increased pH is associated with the lower likelihood of CaOx crystallization, and thus the less risks of nephrolithiasis. On the other hand, the 24-hour urine samples from the subjects of Group 2 did not show a similar pH increase after the Intervention with placebos: the pH remained at 6.5 despite the Intervention.

For both Groups 1 and 2, the 24-hour urine creatinine was not significantly affected by the Intervention.

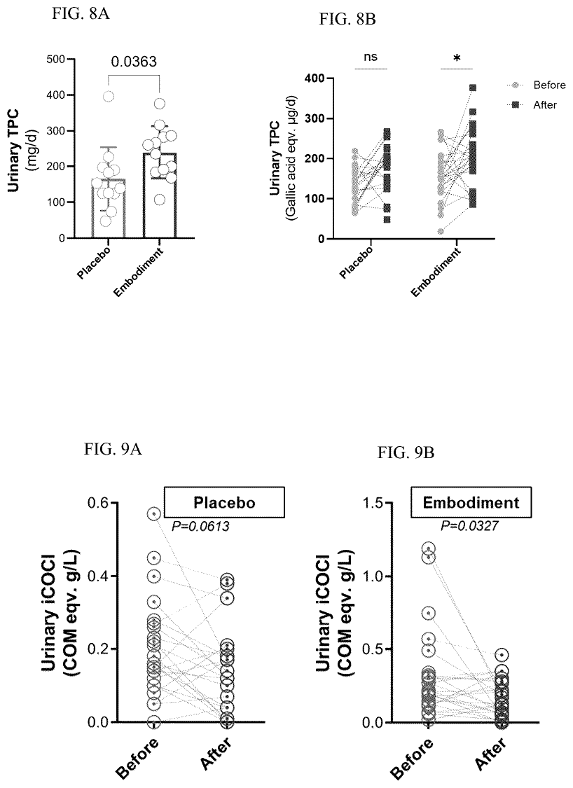

Moreover, total phenolic content (TPC) in 24-hour urine samples of subjects from Group 1 (ingesting embodiments) and subjects from Group 2 (ingesting placebos) after the Intervention (Day 10) were analyzed and compared. Significantly higher urinary TPC was found from the samples of Group 1 subjects than from the samples of Group 2 subjects (FIG. 8A). When comparing the changes occurred to the two Groups as a result of the Intervention (before vs. after the Intervention), the present inventors found that the TPC in the urine samples from Group 1 increased significantly (P<0.05), whereas the TPC in the urine samples from Group 2 did not increase significantly (FIG. 8B). These findings led the present inventors to conclude that ingestion of the embodiments increased the level of antioxidants in the human body, especially antioxidants in the polyphenol group.

Furthermore, the present inventors tested the indole-reacted calcium oxalate crystallization index (iCOCI) in the subjects' 24-hour urine samples, particularly for the changes of iCOCI resulting from the Intervention administered to each Group. As noted earlier, the iCOCI represents the crystallization potential of calcium oxalate (CaOx), which is a strong indication of the subject's likelihood of developing nephrolithiasis (More-Krong et al., Sci Rep 10 (1) (2020) 8334). The results are shown in FIGS. 9A-9B. Following their respective Interventions, the urinary iCOCI of Group 1 (ingesting embodiments) decreased from 0.32±0.31 to 0.17±0.13 COM eqv. g/L at P=0.033, which was statistically significant (FIG. 9B); on the other hand, the urinary iCOCI of Group 2 (ingesting placebos) decreased from 0.20±0.13 to 0.14±0.12 COM eqv. g/L at P=0.061, which was statistically insignificant (FIG. 9A).

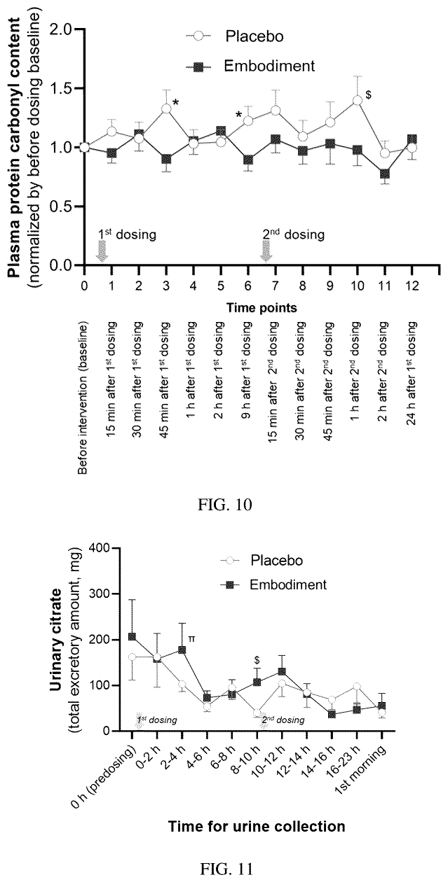

Blood samples from both Groups, collected during the PK study in the early Intervention period (Days 3-4), were tested for the protein carbonyl level in their plasma. Said protein carbonyl level is a strong indicator of the subject's protein oxidation due to the oxidative stress (Dalle-Donne et al., Clinica Chimica Acta, 329 (1)-(2) (2003) 22-38). The results are shown in FIG. 10. The protein carbonyl level of Group 1 subjects (dosing/ingesting embodiments) started decreasing significantly 45 minutes after the first ingestion of embodiment, 9 hours after the first ingestion of embodiment, and 1 hour after the second ingestion of embodiment, and then the trend of decrease continued afterwards. The protein carbonyl level of Group 2 subjects (dosing/ingesting placebos) did not exhibit such trend. This finding led the present inventors to conclude that, as soon as 45 minutes after ingestion, the embodiments according to the present invention was effective in inhibiting the protein oxidation and thus counteracting oxidative stress, thereby addressing a key risk factor of nephrolithiasis.

Furthermore, the urine samples from both Groups, collected during the PK study in the early Intervention period (Days 3-4), were tested for urinary citrate excretion. As noted previously, one key risk factor of nephrolithiasis is low urinary citrate excretion. The results are shown in FIG. 11. In the 2nd hour after the Intervention (i.e., dosing/ingesting either embodiments or placebos), the urinary citrate excretion of Group 1 subjects (dosing/ingesting embodiments) became greater than that of Group 2 (dosing/ingesting placebos) with marginal significance; in the 8th and 10th hours, the citrate excretion of Group 1 became greater than that of Group 2 significantly. Therefore, the present inventors concluded that the embodiments according to the present invention were effective in promoting the urinary citrate excretion, thereby addressing another key risk factor of nephrolithiasis.

### Further Observations

During and after the Trial, no adverse or abnormal effect on the subject's health was observed or reported. The present inventors have therefore concluded that the embodiment is safe for daily consumption by healthy humans.

Thus, the present inventors have ascertained a method for inducing anti-nephrolithiasis effect in a human body by oral ingestion of a beverage prepared from the composition in accordance with an embodiment of this invention.

### Pilot Trials in Human Nephrolithiasis Patients

### Description of the Trial

Pilot Trial, in which the subjects were humans who were diagnosed with nephrolithiasis, was conducted. The screening diagnosis was based upon KUB X-ray and/or CT scan medical image. Eleven volunteering patients, of both genders and 49±13 years of average age, participated in the Trial.

Before the Pilot Trial, urine samples over a 24-hour interval were collected from the patients/subjects.

During the Pilot Trial, the “Intervention” was administered: the subjects were instructed to orally ingest at least 2-3 dosages of embodiments daily for 30 days. Each dosage contained 500 mL of the embodiment and was prepared by mixing 60 mL of a beverage concentrate of an exemplary composition (see Table 1 above) with 440 mL of reverse-osmosis (RO) water, equivalent to a ratio of 0.136 parts of the exemplary composition to one part of water. No placebo was administered to the subjects.

After the trial, urine samples over another 24-hour interval were collected from the subjects.

The samples collected from pre-Trial (Day 0) and post-Trial (Day 30) periods (also called before-Intervention and after-Intervention) were subsequently tested for urine volume, urinary creatinine, urine pH, urine specific gravity, urine electrical conductivity, urine oxalate, urinary calcium, urinary citrate (by high-performance liquid chromatography HPLC), urinary calcium oxalate crystallization index (COCI), urinary indole-reacted calcium oxalate crystallization index (iCOCI), total antioxidant capacity (TAC by ABTS method) and total phenolic content (TPC). If a nephrolith was also found from the subject's any urine (i.e., between Day 0 and Day 30), that nephrolith was further analyzed by Fourier transform infrared (FTIR) spectroscopy.

### Analysis Results

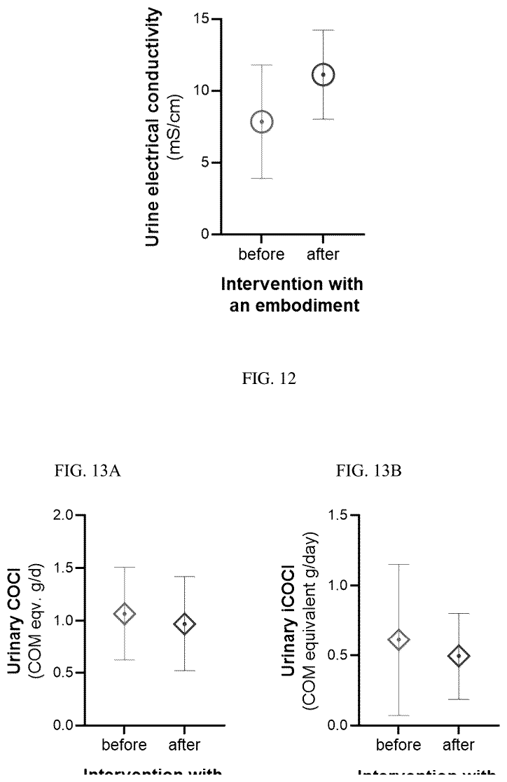

The Trial did not significantly affect the urine volume and urinary creatinine level. As shown in FIG. 12, the after-Intervention urine samples exhibited an increase in electrical conductivity when compared with the before-Intervention samples. This trend is associated with a decreased risk of nephrolithiasis.

As shown in FIGS. 13A-13B, the after-Intervention urine samples exhibited decreases in COCI (FIG. 13A) and iCOCi (FIG. 13B) when compared with the before-Intervention samples. Both these trends are associated with the decreased risk of CaOx nephrolithiasis.

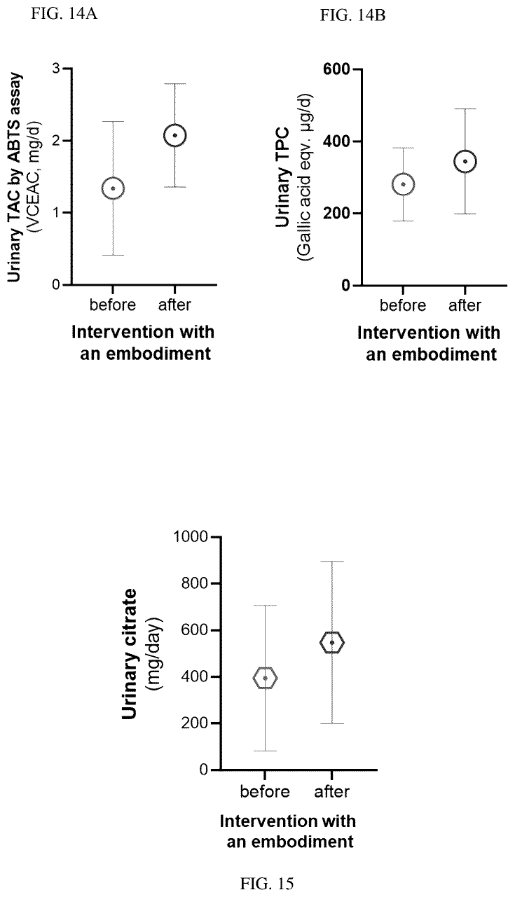

As shown in FIGS. 14A-14B, the after-Intervention urine samples exhibited increases in total antioxidant capacity (TAC) (FIG. 14A) and total phenolic content (TPC) (FIG. 14B) when compared with the before-Intervention samples. These findings support that continued ingestion of embodiments increases the antioxidant levels in the human body.

The after-Intervention urine samples exhibited a slight increase in the urinary calcium and substantially the same level of the urinary oxalate.

As shown in FIG. 15, the after-Intervention urine samples exhibited an increase in urinary citrate level when compared with the before-Intervention samples. High citrate level is a key indication of anti-nephrolithiasis effect.

### Further Observations

Among the 11 subjects in this Trial, 7 subjects were diagnosed with asymptomatic cases of nephrolithiasis; the remaining 4 subjects were diagnosed with active symptomatic cases of nephrolithiasis.

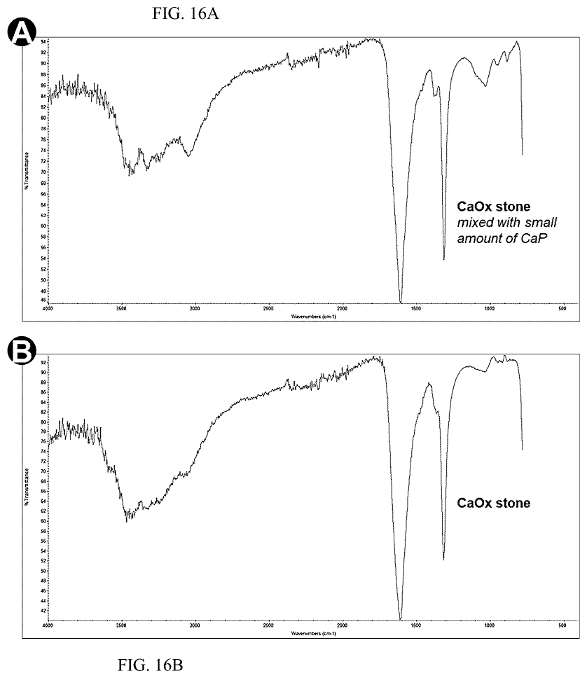

Three out of the four symptomatic subjects reported the diminishment in the sizes of their nephroliths. Said diminished nephroliths were extracted from the subject's bodies through a normal urination taking place between Day 0 and Day 30. Two out of said three reporting subjects were able to collect the nephroliths diminished and extracted thus. The later Fourier transform infrared (FTIR) spectroscopy analysis confirmed that the nephroliths collected from said two subjects were of the CaOx type, one of them being mixed with a small amount of calcium phosphate CaPs. FIGS. 16A-16B show the results of said FTIR analyses performed upon the nephroliths samples.

Further, two out of the four symptomatic subjects reported the improvement of their medical conditions: one reported that during the Trial his nephrolith moved to the urethra in the penis, and then the nephrolith felt much diminished and/or extracted from his body unawares, and thus he would require a surgical operation no more; the other reported that the diminishment of their nephrolith allowed them to undergo a less invasive surgical operation.

Thus, the present inventors have ascertained a method for treating a human nephrolithiasis patient by oral ingestion of a beverage prepared from the composition in accordance with an embodiment of this invention.

### Taste Testing Survey Trials

To ascertain the embodiments' flavor which is one advantageous effect of the present invention, the present inventors have conducted a taste testing survey. Fifteen participants were asked to taste and rate their satisfaction on a scale of 0-5 (0 being the most unsatisfied; 5 being the most satisfied) with the taste of an embodiment.

Embodiments in this survey were prepared by mixing 60 mL of a beverage concentrate of an exemplary composition (see Table 1 above) with 440 mL of reverse-osmosis (RO) water, equivalent to a ratio of 0.136 parts of the exemplary composition to one part of water.

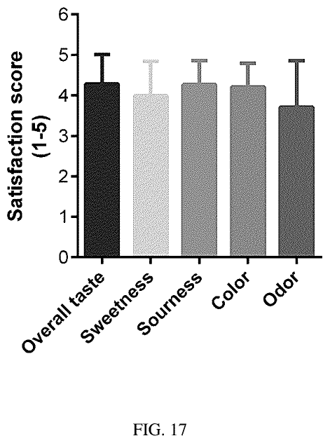

The results of the taste testing survey results are shown in FIG. 17. The average satisfaction ratings were over 4.0 for the embodiment's overall taste, sweetness, sourness and color; the odor was rated 3.7 in average. No participant reported that the embodiment was unpleasant or repulsive. This finding ascertained that the embodiments are conducive to daily and routine consumption, addressing the prior art's problem of non-adherence to the nephrolithiasis treatment or preventive measures, and behaviorally increasing the fluid intake and thereby addressing a key risk factor of nephrolithiasis.

1. A composition of a beverage concentrate having anti-nephrolithiasis effect, said composition comprising: 1-9.99% by weight of citric acid; 60-90% by weight of a water extract of banana stem; 0.01-1.02% by weight of lipoic acid; 0.001-2.005% by weight of rutin; and 0.01-0.65% by weight of at least one natural antioxidant.

2. The composition of claim 1, wherein the lipoic acid is alpha-lipoic acid (ALA).

3. The composition of claim 1, wherein the natural antioxidant has colorant property.

4. The composition of claim 1, comprising 0.01-0.20% by weight of the natural antioxidant, said natural antioxidant being derived from sappan heartwood.

5. The composition of claim 1, comprising 0.05-0.45% by weight of the natural antioxidant, said natural antioxidant being derived from butterfly pea flower.

6. The composition of claim 1, comprising 0.01-0.20% by weight of a first natural antioxidant and 0.05-0.45% by weight of a second natural antioxidant, said first antioxidant being derived from sappan heartwood and said second antioxidant being derived from butterfly pea flower.

7. The composition of claim 1, further comprising 0.01-25.20% by weight of a non-sugar natural sweetener.

8. The composition of claim 7, comprising 0.01-0.15% by weight of the non-sugar natural sweetener, said non-sugar natural sweetener being stevia.

9. The composition of claim 7, comprising 0.01-0.15% by weight of the non-sugar natural sweetener, said non-sugar natural sweetener being sucralose.

10. The composition of claim 7, comprising 14.01-25.20% by weight of the non-sugar natural sweetener, said non-sugar natural sweetener being maltitol.

11. The composition of claim 1, comprising 1.01-5.01% by weight of citric acid.

12. The composition of claim 1, comprising 70-85% by weight of the water extract of banana stem.

13. The composition of claim 1, comprising 0.01-0.05% by weight of lipoic acid.

14. The composition of claim 1, comprising 0.001-1.005% by weight of rutin.

15. The composition of claim 1, further having anti-aging and lifespan-extending effects.

16. The composition of claim 4, comprising 0.05-0.15% by weight of the natural antioxidant derived from sappan heartwood.

17. The composition of claim 5, comprising 0.15-0.35% by weight of the natural antioxidant derived from butterfly pea flower.

18. A beverage prepared from the composition in accordance with claim 1 by mixing the composition with water at the ratio of 0.05-0.15 parts of the composition to one part of water.

19. The beverage of claim 18, prepared by mixing the composition with water at the ratio of 0.10-0.15 parts of the composition to one part of water.

20. A method for inducing anti-nephrolithiasis effect in a human body by oral ingestion of a beverage prepared from the composition in accordance with claim 1.

21. A method of claim 20, wherein at least 1,000 mL of the beverage is orally ingested daily.

22. A method for treating a human nephrolithiasis patient by oral ingestion of a beverage prepared from the composition in accordance with claim 1.

23. A method of claim 22, wherein at least 1,000-1,500 mL of the beverage is orally ingested daily.
